(19) United States
(12) Patent Application Publication
(10) Pub. No.: US 2008/0249633 A1
(43) Pub. Date: Oct. 9, 2008

(54) Biodegradable Materials and Methods of Use

(76) Inventor: Tim Wu, Shrewsbury, MA (US)

Correspondence Address: CHOATE, HALL & STEWART LLP, TWO INTERNATIONAL PLACE, BOSTON, MA, 02110, US

(21) Appl. No.: 11/843,528

(22) Filed: Aug. 22, 2007

Related U.S. Application Data
(60) Provisional application No. 60/823,168, filed on Aug. 22, 2006.

(51) Int. Cl. A61F 2/28; A61F 2/00; A61P 9/00

(52) U.S. Cl. 623/23.58; 424/423

(57) ABSTRACT

The present invention provides novel and inventive biodegradable and biocompatible materials and methods of use in biomedical area. Inventive materials can be formed by blending PLGA with ACP or any one of their family members. Inventive materials can be used in making biodegradable products including but not limit to drug eluting stents, vascular graft, bone substitutes such as bone fixation screws, surgical sutures and anti-adhesive membranes, and/or drug-slow release control vehicle etc.

### CROSS-REFERENCE TO RELATED APPLICATION

This patent application claims the priority of U.S. provisional application No. 60/823.168 filed on Aug. 22, 2006.

### FIELD OF INVENTION

This invention is related to a biodegradable material and methods of formulation and its use in making biomedical products.

### BACKGROUND OF THE INVENTION

### Polyester Biodegradable Material

Polyester biodegradable material is a widely used material in making biodegradable products in the area of bone tissue regeneration, cardiovascular devices, drug delivery vehicles etc. Polyester polymer is a family including polylactides (PLA), polyglycolides (PGA) and their copolymer PLGA etc.

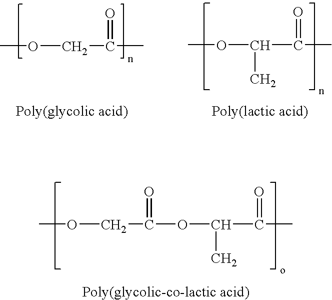

repeat structure of the PLA, PGA, and PLGA biodegradable polyesters.

As well-known biodegradable polymers in medical applications, PLA and PGA are attractive for having lactic acid and glycolic acid as their degradation products, respectively. These natural metabolites are ultimately converted to water and carbon dioxide through the action of enzymes in the tricarboxylic acid cycle and are excreted via the respiratory system. In addition, PGA is also partly broken down through the activity of esterases and excreted in the urine. Along with its superior hydrophobicity, PLA is more resistant to hydrolytic attack than PGA, making an increase of the PLA:PGA ratio in a PLGA copolymer result in delayed degradability. Table 1 is the summary of degradation time among different Poly-lactide and Copolymers.

TABLE 1
Physicochemical characteristics and biodegradable time
of Poly-lactide and Copolymers.

| | | | Bio- |
|---|---|---|---|
| | | | degradation |
| | | Glass | Time |
| Polymer | Crystallinity | Transition | (months) |
| Poly(L-lactide) | Crystalline | 45-60° C. | 18-24 |
| Poly (D,L-lactide) | Amorphous | 50-60° C. | 12-16 |
| 50:50 (D,L-lactide-co-glycolide) | Amorphous | 45-55° C. | 2 |
| 85:15 (D,L-lactide-co-glycolide) | Amorphous | 45-60° C. | 5 |

(* biodegradation time depend on the formulation, porosity, surface area and polymer molecular weight.)

However, the major problems which has slowed-down its application as a widely accepted biodegradable material is its inflammatory response to surrounding tissue caused by acidic products released from polyester material degradation.

### Calcium Phosphates Families

Calcium Phosphates, a family (Table 2) of inorganic biodegradable polymers has been widely applied as tissue engineering scaffold. Due to their uncontrollable degradation rate and poor drug impregnation characteristics, Calcium Phosphates material alone were seldomly used as drug delivery polymer but as a biofiller in biotissue engineering composites.

TABLE 2
The family members of calcium phoshates and their polymers

| Chemical Name | abbr | Chemical Formula | Phase | Ca/P |
|---|---|---|---|---|
| Amorphous calcium phosphate | ACP | | | |
| Dicalcium Phosphate | DCP | CaHPO4 | Monetite | 1 |
| Tricalcium Phosphate | α-TCP | Ca3(PO4)2 | | 1.5 |
| Tricalcium Phosphate | β-TCP | Ca3(PO4)2 | Whitlockite | 1.5 |
| Pentacalcium Hydroxyl Apatite | HAp | Ca10(PO4)6(OH)2 | Hydroxyapatite | 1.67 |
| Tetracalcium Phosphate Monoxide | TTCP | Ca4O(PO4)2 | Hilgenstockite | 2 |

Among the family members, ACP has become increasingly significant in biomaterial tissue engineering. It is an important intermediate product for in vitro and in vivo apatite formation with high solubility and better biodegradability. It was mainly used in the form of particles or powders, as an inorganic component incorporated into biopolymers, to adjust the mechanical properties, biodegradability, and bioactivity of the resulting composites. Based on the similarity of ACP to the inorganic component of the bone, ACP was used as a bioactive additive in several Bis-GMA resin-based dental materials to improve remineralization. Based on its solubility, the corresponding composites were used to release ions into aqueous media, forming a favorable super saturation level of Ca²⁺ and PO₄³⁻ ions for the formation of apatite. The ion release is also considered to having roles in neutralizing the acidity resulted from polymer biodegradation, retarding bioresorptive rate and eliminating inflammation occurrence.

By blending polyester materials with calcium phosphates, we invented a new biodegradable material with high biocompatibility and precisely drug-release controllability.

### SUMMARY OF THE INVENTION

It is therefore a primary object of this invention to provide a biodegradable material, when implanted or administrated, will be absorbed automatically in approximately 1-12 months.

It is a further objective of this invention to provide a biodegradable material that not only prevent inflammatory response to the surrounding tissue, but also can promote the surrounding tissue regeneration (bone implant) or re-endothelization (cardiovascular prostheses).

A preferred embodiment of the material of the invention comprise one of Polyester family member including but not limit to polylactides (PLA), Poly(L-lactide)(PLLA), Poly (D,L-lactide)(PDLA,) polyglycolides (PGA) and their copolymer: Poly(D.L-lactide-co-glycolide) (PLGA) etc.

The preferred PLA/PGA ratio in PLGA copolymer in the said invention is ranged from 1% to 99%

Another preferred embodiment of the biodegradable material comprises one of another biodegradable polymer—Calcium Phosphates family members. The said calcium phosphates family members include but not limit to Amorphous Calcium Phosphate (ACP), Dicalcium Phosphate (DCP), Tricalcium Phosphate (α-TCP), Tricalcium Phosphate(β-TCP), Pentacalcium Hydroxyl Apatite(HA), and Tetracalcium Phosphate Monoxide(TTCP) etc.

Preferred Polyester/Calcium Phosphates ratio in the invented material is ranged from 1% to 50%.

The invented material can be applied but not limited to Cardiovascular Disease such as fully biodegradable drug eluting stent; biodegradable coated drug eluting stent; vascular(including both coronary and peripheral) grafts; Bone Tissue Regeneration such as bone fixation screws, bone substitute; Drug Delivery Vehicle such as vaccine, pain killer; Surgical Product such as sutures, etc.

The said material can be blended through a blender either before the making of final products or applied separately to a product but formed together once the product was finalized.

### BRIEF DESCRIPTION OF THE DRAWINGS

FIG. 1 depicts exemplary microscopic images showing the nano-porous structure of invented material as described in Example 3 and Example 4.

FIG. 2 depicts an exemplary microscopic image from stent surface coated with exemplary inventive material as described in Example 4 and Example 5.

### DETAILED DESCRIPTION OF THE PREFERRED EMBODIMENTS

The preferred embodiments of polyesters include but not limited to PLA, PLLA, PDLA, and PLGA. All these materials are commercial available currently. The proffered material for PLGA copolymer contain PLA/PGA ratio from 1% to 99%. The material can be dissolved in THF, chloroform, or other organic solvents or be blended in a blender without any solvent.

The preferred embodiments for calcium phosphates include but not limited to Amorphous Calcium Phosphate (ACP), Dicalcium Phosphate (DCP), Tricalcium Phosphate (α-TCP), Tricalcium Phosphate(β-TCP), Pentacalcium Hydroxyl Apatite(HA), and Tetracalcium Phosphate Monoxide(TTCP) etc. All these materials are commercially available.

The preferred embodiment for blending two material together include but not limited to direct blending such as using rotary blender and then cast into a final biodegradable product such as bone fixation screws by using a injection mould. Or dissolving both polymers into organic solvent such as THF, Chloroform etc, and then coat on the surface of product such as stents or vascular grafts, Or emission both materials directly to form a micro-particle as drug delivery vehicle such as vaccine, etc.

### EXAMPLES

### Example 1

### Preparing Invented Material

Dissolving PLGA (85/15, sigma) 2 mg and ACP 1 mg in 2 ml THF solution, mixing thoroughly through vortex and then put into 50F water bath for 1 hour, The combined two materials were well mixed and can be applied as coating matrix for drug delivery.

### Example 2

### Preparing Invented Material

Dissolving PLGA 2mg and HA 1 mg into 2 ml chloroform solution as described in example 1, the blended material formed in a cloudy matrix due to the relative insoluble of HA.

### Example 3

### Preparing Invented Material

Dissolving PLGA 2 mg in 1 ml THF, and dispersing 1 mg ACP in 1 ml THF individually, as described in example 1, then combine the two solutions together and mix thoroughly. The ACP was evenly distributed in PLGA polymer.

### Example 4

### Applying Invented Material

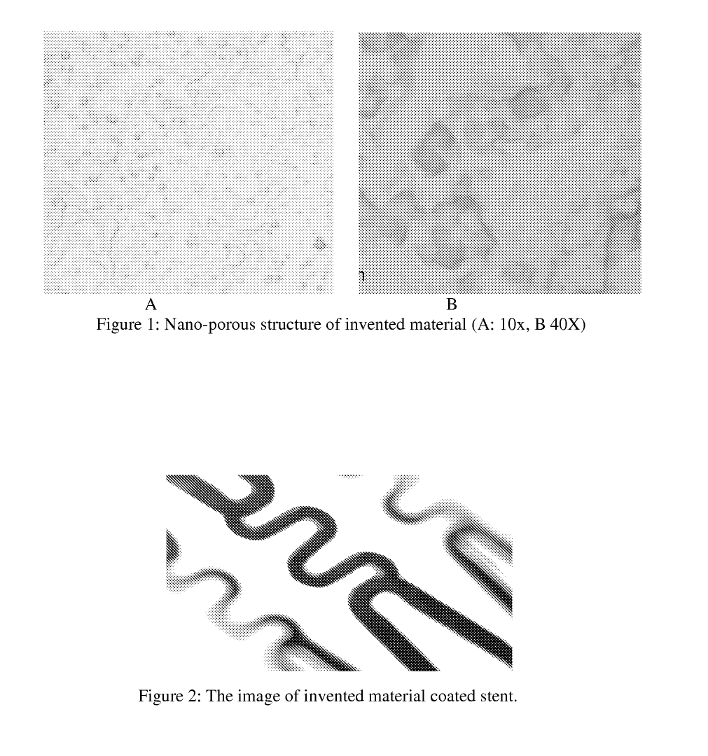

The material produced during Example 3 was spraying-coated on the surface of transparent glass. The microscopic observation showed that the invented material formed in a nano-porous structure as demonstrated in FIG. 1.

### Example 5

### Applying Invented Material

The invented material prepared at example 3 was further spraying-coated on the surface of metal stent. Microscopic observations showed that the material was uniformly coated on the surface of metal stent. FIG. 2 is the image from the invented material coated stent. Further implant these stents into pig coronary arteries for one month shown that the invented material coated stent has less restenosis formation than both PLGA and PEVA/PBMA copolymer coated stents.

Although specific feature of the invention are shown in some drawing and or others, this is for convenience only as some feature may be combined with any or the other entire feature in accordance with the invention.

What is claimed is:

1. A biodegradable composition comprising polyester polymers and calcium phosphates materials.

2. The composition of claim 1, wherein said polyester polymers is Poly(D.L-lactide-co-glycolide)(PLGA) or any of its family members including but not limited to: polylactides (PLA), Poly(L-lactide)(PLLA), Poly (D,L-lactide)(PDLA,) polyglycolides (PGA) etc.

3. The composition of claim 1, wherein said calcium phosphates is Amorphous Calcium Phosphate (ACP) or any of its family members including but not limited to: Dicalcium Phosphate (DCP), Tricalcium Phosphate (α-TCP), Tricalcium Phosphate(β-TCP), Pentacalcium Hydroxyl Apatite(HA), and Tetracalcium Phosphate Monoxide(TTCP), etc.

4. The composition of claim 1, wherein said polyester polymer is PLGA copolymer, the ratio of PLA/PGA in said copolymer is ranged from 1% to 99%.

5. The composition of claim 1, wherein said Calcium Phosphates in the composition is ranged from 1% to 50%.

6. A method of making the composition of claim 1 comprising blending directly or dissolving in organic or inorganic solvent and then applied in a mix or separately.

7. A biodegradable drug eluting stent; biodegradable coated drug eluting stent; vascular(including both coronary and peripheral) grafts; bone tissue regeneration such as bone fixation screws, bone substitute; drug delivery vehicle such as vaccine, pain killer; or surgical product such as sutures, etc. made or coated with the biodegradable composition of claim 1.

8. A method of treating cardiovascular diseases using the biodegradable drug eluting stent; biodegradable coated drug eluting stent; vascular(including both coronary and peripheral) grafts; bone tissue regeneration such as bone fixation screws, bone substitute; drug delivery vehicle such as vaccine, pain killer; or surgical product such as sutures, etc. according to claim 7.
